(19) United States
(12) Patent Application Publication
(10) Pub. No.: US 2010/0028276 A1
(43) Pub. Date: Feb. 4, 2010

(54) SUNSCREEN COMPOSITIONS

(76) Inventor: Guerry L. Grune, Virginia Beach, VA (US)

Correspondence Address: GUERRY LEONARD GRUNE, 784 S VILLIER CT., VIRGINIA BEACH, VA, 23452, US

(21) Appl. No.: 12/449,932

(22) PCT Filed: May 5, 2007
(86) PCT No.: PCT/US2007/005672

§ 371 Date: Sep. 3, 2009

(51) Int. Cl. A61K 8/19; A61Q 17/04; C12Q 1/02

(52) U.S. Cl. 424/59; 435/29

(57) ABSTRACT

Sunscreen and sunblock formulations that provide cytoprotective and immuno-enhancing additives avoiding endocrine disrupting agents, (a) at least one inorganic sun-blocking or sunscreen agent that is a proven non-endocrine disrupter, (b) at least one emollient proven to be a non-endocrine disrupter; and (c) an oil component capable of protecting skin from harmful effects of radiation including sunlight and ultraviolet light wherein the oil is a carrier oil, an essential oil, or both are described. The formulations preferably include an essential oil of a naturally-occurring substance as well as suitable inorganic components including titanium dioxide, zinc oxide, silica or silicon dioxide, fluoropolymers, and mixtures thereof. Suitable organic components include butylmethoxy dibenzoylmethane. Cold-processed Aloe barbadensis Miller-Stockton, a single species of Aloe barbadensis containing glucose-rich mannose-containing oligosaccharide or oligosaccharides, can also be used. Test methodologies and results indicate specific differences from conventional formulations.

### FIELD OF INVENTION

This invention relates to sunscreens and sunblocking agents, generally, and, more specifically, to sunscreens and sunblocking agents which include cytoprotective additives and/or which do not include suspected or documented endocrine disruptive agents. The endocrine-disrupting nature of various active sunscreen or sunblock agents as well as non-active constituents that are used in the formulations, can be measured using a specific test known as a LUMI-CELL™ ER estrogenic cell bioassay system, which is also described herein.

### BACKGROUND

Although a tan has long been considered a symbol indicative of good health, excessive exposure of the human skin to sunlight is harmful. For this reason, many people use sunscreens and/or sunblocking agents to minimize their exposure to harmful rays.

A study by Margaret Schlumph at the Institute of Pharmacology and Toxicology at the University of Zurich first published in 2001, supports earlier health concerns regarding the use of endocrine disrupting organic substances in nearly all UV screening chemicals used in sunscreens. The association between the exposure and bioaccumulation of endocrine disruptor chemicals (EDCs) and their adverse effects on human and wild life populations has raised concern worldwide. Due to the detrimental effects of environmental exposure to EDCs, there is an obvious need to develop a relevant bioassay, which can both detect these chemicals, as well as provide a relevant estimate of their endocrine disrupting potency. Some examples of the effects of EDCs are: decreased reproductive success and feminization of males in several wildlife species; increased hypospadias along with reductions in sperm counts in men; increase in the incidence of human breast and prostate cancers; and endometriosis. Because these chemicals are ubiquitous, highly lipophilic, and often chlorinated, this ensures their persistent presence in the environment resulting in their bioaccumulation in the food chain.

It would be advantageous to provide sunscreen and sunblock formulations which do not include such endocrine disrupting agents. In addition, many of the non-active components or constituents also have been found to be endocrine disrupters and therefore it would be advantageous to prepare formulations with both active and non-active ingredients that are free from endocrine disrupting compounds or agents. It would also be advantageous to provide cytoprotection to the skin using cytoprotective agents in the formulations. In addition, the use of immuno-enhancing compounds or agents is beneficial for sun protection formulations. The present invention provides such sunscreen and sunblock formulations.

### SUMMARY

### Compositions:

The composition of one embodiment contains at least the following components:
(a) an inorganic sun-blocking non-endocrine disruptive sunscreen agent such as micronized zinc oxide or titanium dioxide with particle sizes in the 20-300 nm range
(b) a non-endocrine disrupting cytoprotective emulsifier or emulsifier mixtures;
(c) an oil component comprising a carrier oil, preferably an essential oil any of which are also non-endocrine disruptive and;
(d) at least one emollient, where the emollient may also be the cytoprotective emulsifier of (b) above
(e) sun boosting additives that are non-endocrine disruptors—especially silicone oils that are D5 based (Si—O— cyclics) and linear silicone oil based chemistry either alone or in combination with essential oils.

Sunblock and sunscreen compositions that are essentially free of endocrine disrupting agents, which provide cytoprotection to the skin, and are immuno-enhancing, are disclosed. In all embodiments, the compositions provide protection in the UV-A and UV-B long range and short range ultraviolet radiation spectrum. Ideally, the compositions provide coverage that is easily maintained, for example, by being waterproof and perspiration proof, and also easily applied and convenient to use, for example, invisible or nearly invisible once applied to the skin, non-staining and non-greasy. Further, the compositions ideally do not cause irritation.

Advantageously, both active and inactive sun-block or sunscreen ingredients are void of any known or suspected endocrine disrupters.

Another embodiment is directed toward a colored sunscreen comprising: (a) at least one ultramarine pigment that imparts a color other than white to the emulsion with a titanium dioxide or zinc oxide or possibly fumed or fused silica or even silicon dioxide (b) at least one sunscreen active agent in an amount effective to protect skin against the actinic radiation of the sun—this preferably being ZnO or Z-Cote (micronized particles—preferably nanoparticle sized to assure transparency); (c) no known or suspected endocrine disrupting organic substances; (d) a cytoprotective substance such as a glucose-rich mannose-containing oligosaccharide obtained from and used with aloe barbadensis Miller as an emulsifier; and (e) sufficient water to form other than a white colored emulsion; and sufficient dispersion to assure SPF of at least 15 and an SPF booster that itself is not an endocrine disrupter and shows no appreciable toxicity.

The amount of the ultramarine pigment in the composition can range form about 0.5 to 10 weight percent of the composition, preferably form 1 to 5 weight percent of the final formulation.

Optionally, the colored sunscreen emulsion can contain one or more additional ingredients, including emollients, waterproofing agents, dry-feel modifiers, insect repellants, antimicrobial preservatives and/or fragrances

In another embodiment, the compositions comprise known cytoprotective and immuno-enhancing oligosaccharides from aloe barbadensis Miller. The compositions can enhance the immuno-responsiveness of skin cells to UV light by using extracts of aloe or similar naturally-occurring substances, such as kukua nut extract and other similar naturally-occurring anti-inflammatory substances. In this embodiment, the substances are not processed, unless the beneficial immino-responsive effects are not lost during processing. Ideally, if processed, the substances are processed within a short time period (i.e., a few days) after harvesting, and the plants and subsequent extracts are kept cool (at or below room temperature) during processing. The immunoenhancing and cytoprotective agents are obtained from “cold-pressed” aloe which contains the beneficial oligosaccharides and provides an emollient base for the formulation. Other cytoprotective and immuno-enhancing agents, such as amino-acids, vitamins or pro-vitamins, nucleo-derivatives such as nucleosides or nucleotides and/or polymers thereof and vegetable extracts, can also be used. Amino acids that can be used include, for example, tryptophan, histidine, phenylalanine, and tyrosine. Vitamins and provitamins include vitamin B6, vitamin A, vitamin E, tocopherols, betacarotene, bioflavonoids, as well as the afroementioned nucleotides and polymers thereof. Essential oils and plant extract include cascara, frangula, camomile, hyperic, calendula, elicriso, licorice or essential oils thereof, as well as the essential oils of frankincense and rosemary.

The compositions of this invention provide formulations having an SPF of at least 10 with a concentration level of titanium dioxide or zinc oxide or a combination of the two with or without silica or silcon dioxide and either with a treated or untreated hydrophilic surface of at least 4% and preferably reach SPF 15 or greater using 14% by weight of the inorganic sunblcoking substituents. The compositions of this invention exhibit extremely efficient uses of sunscreen components, particularly zinc oxide. Alternatively, higher levels of preferably micronized titanium dioxide or zinc oxide can be used if ultramarine pigments are added to the composition. These pigments are known to eliminate the whiteness and poor spreadability of known compositions. For the purposes of this invention, however, these pigments must be known to be non-endocrine disruptive as well as to not interfere with the cytoprotective influence of the oligosaccharide aloe extract. There are several ingredients that contribute to the unexpectedly high efficiency of the compositions blocking of UV radiation. It has been found, however, that only one known organic UVA protector, butyl-methoxydibenzoylmethane has been shown to be benign regarding activity in cells or developmental effects on animals.

The compositions may by necessity, include one or more of a select group of anionic emulsifiers. In particular, salts of certain fatty acids are useful in the formulations of this invention, preferably salts of saturated fatty acids and/or salts of straight-chain fatty acids. Alkali metal salts, alkali earth metal salts and amine salts are more preferable for use in the compositions. For example, stearic acid and its salts are useful as emulsifiers in the compositions. More particularly, the following anionic emulsifiers could be useful in the compositions: sodium stearate, sodium lauryl sulfate, DEA cetyl phosphate, sodium dioctyl sulfosuccinate and the like. Most preferably, the emulsifier should be sodium stearate. While it is not completely understood why some salts of fatty acids result in an improved composition, it is theorized that salts of straight-chain fatty acids, (the fatty acids having a relatively high melting point, above 70° Celsius or higher), are preferable due to their structure. For example, salts of branched or unsaturated fatty acids are most likely not acceptable for use in the compositions. These emulsifiers have not been required in the current formulations but perhaps would be useful if the silicone oils or cocoate esters are not perceived as the best alternatives.

The anionic emulsifiers should be present in the compositions in an amount from about 0.01 to about 10%, more preferably 0.1 to about 7% and most preferably from about 0.5 to about 5%. There may be additional emulsifiers present, such as nonionic emulsifiers known to those of ordinary skill in the art such as sorbitan esters and ethoxylated sorbitan esters, ethoxylated fatty acids, fatty alcohols and ethoxylated fatty alcohol's, fatty glyceride esters and ethoxylated fatty glyceride esters and the like.

However, there may have to be at least one anionic emulsifier present in order to achieve the products associated with the compositions described herein. The fatty acid salt emulsifiers may be added to the composition as the salts, or the salt may be formed in situ.

In the case where salts of fatty acids are used, care should be taken to keep the pH of the compositions at a level above about 5, more preferably, above about 5.5. Maintaining the pH at this level will ensure that these anionic emulsifiers remain in the salt form, which is important in retaining the stability and efficacy of the composition.

Preferably, the carrier oil which is more preferably an essential oil, should be present in the composition in an amount of between about 0.1% and about 10%. More preferably, it should be present in the amount of between about 1% and about 5%. Most preferably, it should be present in the amount of between about 2% and about 3%.

For conventional UV-protection formulations, if there is an oil phase should contain at least two materials, the carrier oil or essential oil and a conventional emollient known to those of ordinary skill in the art as useful in sunscreen products, such as mineral oils, ester oils, vegetable oils, silicones, synthetic emollients such as fatty acid esters and the like. For the present invention, the use of a cold pressed aloe barbadensis Miller and specifically the Stockton species is to be substituted as an emollient or can be used in combination with the oils or synthetic emollients that are proven to be non-endocrine disrupting as well as not interfering with augmenting the cytoprotective enhancing effects of the known effective oligosaccharide aloe extract. The emollient should be present in the formulation in a ratio to the carrier concentration of from about 1:1 to about 3:1, most preferably, about 2:1. The carrier oil and the emollient should compose from about 2% to about 20% of the total composition weight.

The use of aloe as both an emollient and a surfactant/dispersion agent together with either micronized ZnO, titanium dioxide, silicon dioxide, PTFE or other fluoropolymers, silica, etc. (inorganic or acceptable organic sunblocking agents) in the manner outlined above is unique and novel. The addition of silicone oils that are neither toxic or endocrine disrupters or other SPF boosting agents that meet the same requirements is also unique to this invention and has heretofore not been seriously considered or explored.

The compositions of the embodiments described can be incorporated into various cosmetic and personal care products such as hand and body lotions, oils, ointments, lip balm products, facial cosmetics and the like. The base formulations may also be used as carrier compositions for active topical agents having dermatological effects, including depigmentation agents, anti-aging ingredients, antifungal agents, antimicrobial agents, insect repellents and the like. For example, depigmentation agents can include magnesium ascorbyl phosphate or hydroquinone but only used in the final composition if these agents are shown not to be endocrine disrupters per the testing criteria established herewithin. Anti-aging agents can include retinoid compounds and alpha-hydroxy acids again only if these agents are shown not to be endocrine disrupters. Anti-fungal agents that can be included in the compositions of this invention include azole compounds including ketoconazole and the like again only if these agents are shown not to be endocrine disrupters. Anti-microbial agents include triclosan, an unknown agent regarding cytotoxicity or endocrine disruption function. Insect repellant fragrances can be included in the compositions of this invention again only if these agents are shown not to be endocrine disrupters.

An element which should be present in all compositions and embodiments is an inorganic sunscreen compound, such as titanium dioxide, zinc oxide or combinations thereof. Possible other inorganics include the use of fused or fumed silica or even silicon dioxide. Preferably, titanium dioxide and zinc oxide and silica or silicon dioxide should be used having a primary particle size from of less than about 300 nm in diameter. It should be present in the composition in the amount of from about 2% to about 25%. More preferably, it should be present in the amount of from about 2% to about 15%. Most preferably, it should be present in the amount of from about 3% to about 10%. The inorganic sunscreen compound should be oil dispersible, and may be present with or without surface coating.

The ratio of titanium dioxide or zinc oxide to the weight of the carrier oil and the emollient combined should be from about 0.3:1 to about 1:1. Most preferably, the ratio should be between about 0.5:1 and 2:3.

### Method of Making

It has been determined that a dispersion of ZnO or combinations of ZnO, TiO₂and/or silica or silica dioxide can be accomplished using aloe barbadensis Miller by first blending the inorganic particles (micronized or otherwise) at a high speed with for example, a Waring blender, followed by the addition of vegetable glycerin or suitable other products including cocoate esters (derivatives of coconut oil) or other oils to ensure complete dispersion and high speed mixing with for example an IKA mixer at speeds up to an including 2000 rpm. A dispersion without the cocoate ester has shown no known endocrine disrupter concentrations as determined by the LumiCell methodology.

To provide the much needed SPF/IRF/NED dispersion using the method outlined herein is critical to the invention and critical to the needs of an industry which is striving to make an “all natural” sunscreen or sunblock formulation. The industry currently formulates using “pre-fabricated” dispersions in that the dispersions are normally purchased from a secondary source and mixed in with existing lotions, pastes, cremes, etc. This technique is unacceptable and teaches away from the present invention, in that the dispersions themselves contain endocrine disrupters and generally toxic (cell killing) chemicals. Manufacturers using such a technique should not claim an “all natural” composition, and certainly not an endocrine-disrupter free composition.

It has also been determined that it is quite difficult, if not impossible, using current dispersion systems for micronized TiO₂ZnO, SiO₂ and the like, that are endocrine-disruptive free. As discussed below, the endocrine disrupters in the Lumi-cell test technique have been found to kill cells. Therefore, in essence, using one of several definitions of toxicity—the killing of cells or adverse effects occurring as a result of repeated daily dosing of a chemical or exposure to the chemical, for part of an organism's lifespan—the dispersions themselves are toxic.

A particular embodiment includes the use of aloe, not only as an emollient, but also as a very effective dispersing agent for the inorganic micronized (and larger) sunblock active agents. High speed shearing (accomplished in a Waring blender for example), followed by high speed mixing (up to 2000 rpm with an IKA mechanical stirrer for example) provides a consistent, usable, and easily blendable inorganic/organic dispersion free of any known toxic substances (if the aloe source and inorganic particle source is well documented and controlled). The dispersion is essential in providing sufficient homogeneity and SPF values with any associated non-active cream, lotion, gel, spray, etc. that is used to provide a formulation consistent with the basis of the present invention.

To provide the proper SPF value, it is also necessary to enhance or boost the SPF number using boosting agents. These may also be endocrine disrupters or toxic (cell-killing) or both and it has been discovered that at least one silicone oil—Dow Coming 1401—(40-70% Decamethylcyclopentasiloxane and 30-60% octamethylcyclotetrasiloxane) is also essentially non-toxic (in terms of killing cells) and non-endocrine disrupting as shown below. The SPF boosting capabilities of silicone oils has been documented and known, but the ability to determine the associated toxicity or estrogenic potential or endocrine disrupting ability has never before been understood or tested. It is likely that other silicone oils and perhaps derivatives of other natural occurring substances that can provide dispersion capabilities to enhance SPF—such as cocoate esters (derived from coconut oil) or other essential oils may be determined to be free of endocrine disrupting capabilities.

As stated above, recent studies have confirmed the suspicion that endocrine disrupting agents exist in currently available sunscreen formulations including; benzophenone-s, homosalate, 4-methyl-benzylidene camphor, octyl methoxycinnamate, and octyl-dimethyl-PABA. All of these substances, in fact, made cancer cells grow more rapidly and three caused developmental effects in animals. Therefore a. non-endocrine disrupting UV protective formulation should include the use of inorganic sun-block agents, such as titanium dioxide and/or zinc oxide. A recent development in the reduction of particle sizes of ZnO has resulted in microfine essentially clear ZnO when applied to the skin. Formulation in the family known as Z-Cote which is a trademarked composition sold by BASF is one such example of a micronized zinc oxide available today.

Therefore the ultimate UV-protective formulation would safely block or screen UV light, enhance the immune responsiveness of the skin in the absence or presence of UV, and ensure the user that there is no endocrine disrupting substance present so that immuno-reponsiveness is not impaired in the presence of UV light.

One method of making the sunblocking agent inorganic/organic dispersion is as follows:

1In a blender (waring or large manufacturing grade) add at least 14% by weight of micronized ZnO (with or without surface modification and with or without titanium dioxide or silica or silicon dioxide) into at least 24 ounces of pure cold pressed aloe barbadensis Miller (and preferably the single species—Aloe Barbedensis Miller—Stockton) and blend for at least one hour. It is preferred to keep the blending at or below room temperature so the aloe efficacy does not degrade.
2Either while blending, or subsequent to that, optionally a silicone oil or essential oil or both can be added for SPF boosting. Because of “D4” issues a D5 component silicone oil such as Dow Corning 1501 or even the linear Dow Coming 2-1184 silicone oil that has the same volatility as the D4 compounds may be even less toxic and perform well as SPF boosting agent. For the later 2 components there are no known toxic issues and the data for these are readily available from Dow Corning.
3After blending, the dispersion should be transferred to a high speed mixing facility capable of 2000 rpm and mixed for another minimum of 1 hour. At this point the dispersion can be mixed with a preformulated and mixed lotion, creme, paste, etc. that will now incorporate the addition of the high SPF dispersion.
4Blending should also be accomplished at or below room temperature in a chilled vessel if possible. Again, it is desirable to keep the polysachharides and other biologically active and healthful components of the aloe from degrading in the presence of the heat of mixing.

The final formulation can then be transferred to any suitable container and preferably refrigerated until distributed for use.

The formulations of this invention may be prepared using one of at least two methods: a two-vessel method, in which the oil and aloe or water phases are individually prepared, and a one-vessel method into which all ingredients are added in selected specific order. Any of these processes that will produce a smooth uniform, white to light ivory emulsion are satisfactory as long as the inorganic particles are sufficiently dispersed to provide adequate SPF values. When combined with ultramarine pigments, the color will change and also provide a clear appearance (using the micronized inorganics) as the composition is applied to the skin.

In accordance with the two-vessel process, an aloe or water phase is prepared by measuring deionized water into a beaker and mixing. The elements of the water phase, including emulsifiers and humectants, chelators, thickeners, waterproofing agents, neutralizing agents and antioxidants should be added and the solution heated. If an anionic emulsifier is used it may be placed into the water phase or into the oil phase, depending upon the nature of the emulsifier. The oil phase is prepared separately in another vessel, including the anionic emulsifier, carrier oil, emollient and inorganic sunscreen agent. The two phases should then be held at a relatively low temperature and mixed.

In the one-vessel process, the aloe-water and oil phases may be made in the same vessel, provided that the components are added in an appropriate order. For example, the aloe-water phase should be created first, adding water and aloe and ZnO or other sub blocking inorganic additives and optionally certain emulsifiers which are compatible with the aloe-water phase to the vessel. The oil phase components may be added, including, optionally, anionic emulsifiers if they are oil phase compatible and the carrier oil, as well as any additional oil-phase emulsifiers, antioxidants and/or emollients that may be desired and if necessary, with additional ZnO or TiO₂, etc. The temperature should be kept at or below room temperature and should be maintained at this level for about 15 minutes—but longer period of stirring, mixing and blending are desirable. After cooling the pH may then be checked and adjusted if needed. Essential oils may also be added later in very small amounts to provide fragrance of most any naturally occurring plant, crop, fruit, or nut. The essential oils are often obtained by simple distillation.

It should be emphasized that SPF values of 15 or greater can be achieved solely by blending and subsequent mixing of aloe with vegetable glycerine (or glycerol as it is also known) and that we have achieved a superior product using this technique. This would be the so called “aloe-water” phase that would be subsequently mixed at high speed with the so-called “oil-phase”Blending would be accomplished using only the aloe-water phase and in so doing, the aloe would not be necessarily diluted with water until after the full addition and blending of the inorganic sunblocking agents. Water dilution during or after blending is acceptable but not necessary and in some cases it may be undesirable.

### Testing and Rating Systems;

One current measure of effectiveness of a sun protective product is indicated by its sun protection factor (SPF). The sun protection factor is the ratio of the amount of exposure (dose) required to produce a minimal erythema reaction in protected skin to the amount required to produce the same reaction in unprotected skin. The absolute dose differs for each individual. Some essential oils may also provide SPF boosting capabilities as included above.

The compositions and resulting formulations described herein not only protect the wearer from the harmful effects of the sun, but actually strengthen the wearer's ‘neuro-muscular response’. One test method, ‘Applied Kinesiology’has been used to test a user's neuro-muscular response to sunblock formulations. Applied kinesiology (AK) is a form of diagnosis using muscle testing as a primary feedback mechanism to examine how a person's body is functioning.

A more complete rating mechanism than the SPF rating method is suggested here. The immuno-response rating system could be a simple 0-10 value, with 10 applying to a substance within the UV-protective composition that is most beneficial to boosting skin cell immune responsiveness to carcinoma, melanoma, etc. (for instance).

A UV-protective formulation or composition that may inhibit normal endocrine function(s) is at least undesirable, and at most a potential health threat to millions who continue to apply such a formulation or composition directly to their skin. Although the SPF value may be high, the potential for endocrine disruption from existing formulations utilizing higher concentrations of active sunscreen agents may also be high and again this poses the possibility of another ranking system.

The continued and growing concern regarding estrogenic potency of sunscreens and their components associated (non-active) components has led to recent studies reviewing the “active” components of sunscreens such as 3-(4-methylbenxylidene) camphor (4-MBC), Octyl-Methoxycinniamate, and Benzophenone-3 have shown them to be highly estrogenic in assays such as uterine wet weight, cell height, and cell proliferation assays. Studies by Janjua et al. (2004) have shown these compounds in urine and blood plasma after topical application. Janjua et. al. (2004) also found changes in hormone (estradiol and testosterone) levels of participants after topical application. In a recent study conducted while further developing this patent application, several sunscreens currently marketed as well as the “non-active” sunscreen components were tested for estrogenic potency (or endocrine disruptive potential). The popular sunscreens tested include: Coppertone SPF 8; Coppertone SPF 15; Coppertone SPF 30 (Endless Summer); Banana Boat SPF 15; Banana Boat Kids SPF30; Hawaiian Tropic SPF 8; Coppertone Water Babies SPF 45; Banana Boat Baby Magic SPF 50; Hawaiian Tropic Baby Faces SPF 50+and 3^{rd} Rock Sunblock™ SPF 30The 3^{rd} Rock formulation conforms to the requirements described in the present invention. The “non-active” components are compounds used in sunscreens and sunblocks that do not directly protect from UV damage and these include: Lexorez 200 (for dispersion and water resistance); ABIL Wax 9801 (emollient and improves SPF response); TEGO care PS (emulsifier); ABIL WE-09 (Emulsifier—that may boost SPF); KOBO CM3K40T4 (boosts SPF); Lanol 84D Dioctyl malate (allows for smooth texture—stabilizer); Dow Corning 344 (Lubricant and dispersant); Dow Corning 1401 (Lubricant. Both are silicone oils.

In ranking potential endocrine disruption substances, again the 0-10 rating is useful with 0 being the desired criteria that a consumer would want to purchase to ensure consumption of a quality product that is also completely safe in terms of potential adverse health effects regarding the endocrine disrupters.

Based on the results shown in FIGS. 1 and 2 and described below, regarding the LumiCell technique and testing, a logarithmic-type scale is proposed as follows;

01=a β-estradiol equivalent of less than 10 ng/g

2-3=a β-estradiol equivalent of greater than 10 ng/g but less than 30 ng/g

4-5=a β-estradiol equivalent of greater than 30 ng/g but less than 50 ng/g

6-7=a β-estradiol equivalent of greater than 50 ng/g but less than 70 ng/g

8-9=a β-estradiol equivalent of greater than 70 ng/g but less than 90 ng/g

10=a β-estradiol equivalent of greater than 100 ng/g

Therefore, also as part of the present invention, a rating system for UV-protective compositions is proposed that includes;

SPF value—15 or greater desired

Immuno-responsiveness factor (IRF)—5 or higher desired (greater than 0)

Non-endocrine disrupter factor (NED)—0-1 desired

All earth grown ingredients in the composition and resulting formulation

The immuno-responsiveness factor (5 or higher) depends on the concentration of immuno-enhancing ingredients or components in the composition and resulting formulation. It is possible to quantify these constituents either during or after formulation is completed. The simplest technique is to quantify these components before formulation is initiated, when the composition has been finally decided upon.

This rating system has particular relevance to the newly discovered methods reported here required to process a dispersion capable of ensuring an SPF 15 or greater value without sacrificing the need to retain an “all earth grown” or “all natural” composition.

The foregoing examples serve as illustrations of the compositions of this invention, however, they do not limit the scope of the invention described herein.

### BRIEF DESCRIPTION OF THE FIGURES

FIG. 1 is a plot illustrating the estrogenic potential concentration of popular sunscreens and a sunblock using an β-estradiol equivalent of nanograms per gram (of sunscreen) (ng/g) scale.

FIG. 2 is also a plot illustrating the estrogenic potential concentration for “non-active” sunscreen components using a β-estradiol equivalent of nanograms per gram (of the inactive) (ng/g) scale.

### DETAILED DESCRIPTION OF THE FIGURES

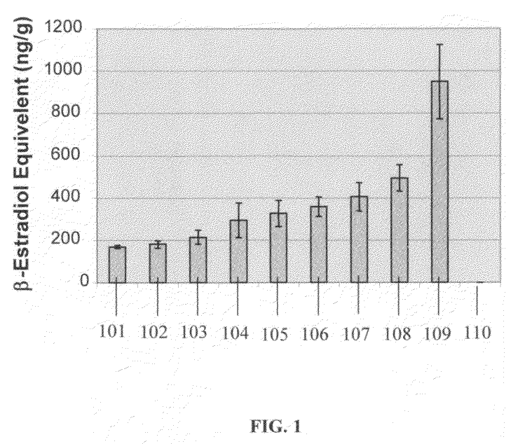

Shown in FIG. 1 is a plot of the estrogenic potential of popular sunscreens measured using β-estradiol equivalents (ng/g). The popular sunscreens that were tested are Coppertone SPF-15™ [101]Hawaiian Tropic SPF-8™ [102]Hawaiian Tropic Baby Faces 50+™[103]Coppertone. SPF-30 (Endless Summer)™ [104]Coppertone SPF-8™ [105]Banana Boat 15™[106]Banana Boat Baby Magic 50™ [107]Banana Boat Kids 30™ [108]Coppertone Water Babies 45™[109] and 3rd Rock Sunblock™[110]In all cases, except 3^{rd} Rock Sunblock™ [110]there was a measurable and significant β-estradiol equivalent.

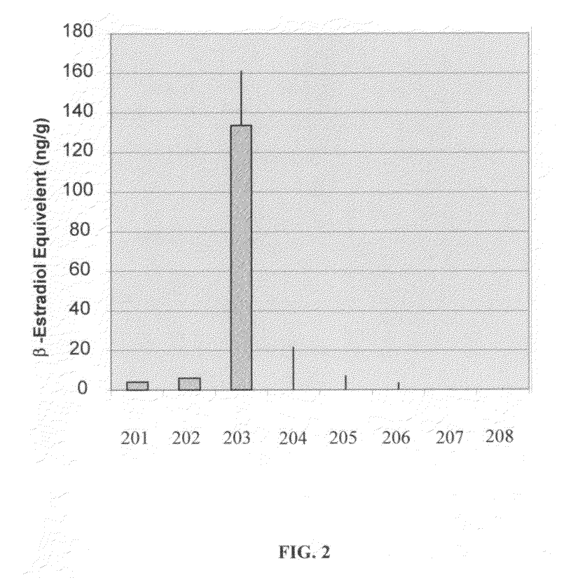

Shown in FIG. 2 is a plot of the estrogenic potential of “non-active” sunscreen chemical components also measured in β-estradiol equivalents (ng/g). The “non-active” components that were measured are Dow Corning 344™ [201] a cosmetic dispersant that is primarily cyclopolydimethylsiloxane. Other components are ABIL Wax 9801™ [202]an emollient; dispersing agent Lexorez 200™ [203]which is a trimethylpentanediol/adipic acid/glycerin crosspolymer; ABIL WE-09™ [204]an emollient; Tego Care PS™ [205] a methyl glucose sesquistearate emollient; Lanol 84D™ [206] which is a stabilizer comprised primarily of dioctyl malate; KOBO CM3K40T4™ [207] which is a composition of cyclopentasiloxane, titanium dioxide, PEG 10 dimethicone, alumina and methicone used primarily as an SPF booster; and Dow Corning 1401™ [208]a dispersant fluid of cyclomethicone and dimethiconol that also has SPF boosting properties.

### EXAMPLES

### Suitable Testing for Endocrine Disruption

In May of 2002, Xenobiotics Laboratories (XDS) of Durham, N.C. submitted preliminary data to ICCVAM for review as a validated regulatory method using their LUMI-CELL™ ER bioassay in response to the Federal Register Notice (Vol. 66, No. 57/Friday, Mar. 23, 2001) as a HTPS method for estrogen active compounds¹⁰. In March of 2004 SACATM gave the LUMI-CELL™ ER bioassay a high priority for validation. In April 2004 the final report on the assay was given to ICCVAM. In March 2005, ICCVAM entered the LUMI-CELL™ ER bioassay into a double blind international validation study using one lab in the European Union, Japan, and the United States. Next, studies were undertaken in which XDS's LUMI-CELL™ ER estrogenic cell bioassay system was used for high throughput screening (HTPS) analysis sunscreens. The results demonstrate the utility of XDS's BG1Luc4E₂ LUMI-CELL™ ER bioassay HTPS system for screening cosmetics for estrogenic/antiestrogenic activity.

There has been a growing need for a fast, reliable, inexpensive method to detect EDCs (endocrine disrupters) in the environment. As part of the present invention, we report a fast, reliable, relatively inexpensive high throughput cell based recombinant bioassay screening method (LUMI-Cell™ ER bioassay) to determine the level of xenoestrogenic EDCs for any cosmetic creme, lotion, paste, etc.

Sunscreen components were purchased from the Inolex Chemical Co., Goldschmidt Chemical Corp., Kobo Products Inc., and Dow Corning. Sunscreens were purchased at Wal-Mart.

LUMI-CELL™ ER Bioassay. The BG1Luc4E2 cell line was constructed as previously described by Rodgers and Denison (2000). Briefly, BG1 cells were stably transfected with an estrogen-responsive luciferase reporter gene plasmid (pGudLuc7ere) and selected for using G418 resistance⁹.

Cell Culture and Bioassay Plates. BG1Luc4E2 cells were grown in RPMI 1640. The cells were transferred into flasks containing DMEM media (supplemented with 5% carbon stripped fetal calf serum and G418 sulfate solution), and incubated for four days before harvesting for BG1Luc4E₂bioassay plates. The cells were then plated in 96 well plates and incubated at 37° C. for 24-48 hours prior to dosing.

Endocrine Extraction Procedure: One gram of each of the lotion components and 0.5 g of each of the sunscreens was placed in MeOH rinsed scintillation vials. Two and 4-gram aliquots of the 3^{rd} Rock Sunblock were also tested. Twenty ml of MeOH was added to each scintillation vial and sonicated for 20 min. Fractions of these extractions, ranging from 1:10 to 1:80,000 were tested. Recoveries were determined using 10ng 17β-estradiol spiked into 3^{rd} Rock Sunblock prior to extraction with 20 ml MeOH compared to 10ng 17β-estradiol spiked into 20 ml MeOH.

Bioassay Dosing Process. Once the assay plate completed its incubation, the media solution in each well was removed and two hundred microliters of DMEM containing the indicated concentration of the desired chemical to be tested was added to each well. The plate was then incubated for 20 hours before analysis of luciferase activity.

Bioassay Analysis by Berthold Luminometer. After lysing the cells (Promega lysis buffer), the luciferase activity was measured in a Berthold Orion Microplate Luminometer, with automatic injection of 50 microliters of luciferase enzyme reagent (Promega) to each well. The relative light units (RLUs) measured were compared to that induced by the 17beta-estradiol standard after subtraction of the background activity. Each compound was tested at least three times on three different sets of plates and the EC50 value in mmol/ml was determined using the Microsoft Excel Forecast function.

To ensure that our claims have scientific basis and merit, 13 sunscreen products and 8 “non-active” lotion components were tested for estrogenic potency. The samples were tested at 4 g, 2 g, 1 g, 0.5 g, and 0.1 g. The 0.5 g aliquot was selected for sunscreens and 1 g for “non-active” components due to it showing the most activity with the least toxicity. The 3^{rd} Rock Sunblock SPF 30™⁽¹¹⁾ was used as a negative control due to it previously testing as a non-detect. The 3^{rd} Rock Sunblock SPF 30 was also used in recovery determinations. This was performed by dividing the average RLU for the 10ng 17β-estradiol spiked 3^{rd} Rock Sunblock SPF 30 by the 10ng 17β-estradiol spiked into 20 ml MeOH. The average recovery was found to be 77.4%.

All of the sunscreens detected positive for estrogenic activity with the exception of 3^{rd} Rock Sunblock, which was shown as a non-detect at less than 0.308 pg/g 17β-estradiol equivalent. The sunscreen with the highest estrogenic potential was Coppertone Water Babies SPF 45 at 948.66±176.62 ng/g 17β-estradiol equivalent. Based on our test results, the order of estrogenic potency appears to be: Coppertone Water Babies 45> Banana Boat Kids 30 > Banana Boat Baby Magic 50 > Banana Boat 15 > Coppertone SPF 8 > Coppertone SPF 30 (Endless Summer) >Hawaiian Tropic Baby Faces 50+> Hawaiian Tropic SPF 8 > Coppertone SPF 15 > 3^{rd} Rock Sunblock SPF 30These results are graphically depicted in FIG. 1

Only 3 of the “non-active” components showed any activity with only Lexorez 200 showing any significant estrogenic potency. The others showed very high detection limits due to their toxicity. These results are summarized and depicted in FIG. 2

It has therefore been demonstrated that the “non-active” components contribute to a portion of the estrogenic potency of many sunscreen formulations. However, a significant portion of the estrogenic potency remains attributed to the “active” components of the same formulations. Further investigations that include testing “active” and “non-active” components for more detailed analysis regarding estrogenic potency ratios are anticipated. It is apparent from the foregoing results that the test methodology enables one to determine the estrogenic potency of any skin product, not just that of one designed for sun protection. It is known that lotions, shampoos, cleansing agents, cremes, sprays, etc. for human and animal skin contact for various uses, contain numerous endocrine disrupting components. Therefore, these embodiments include a test methodology to determine levels of toxicity (as defined by killing cells) that determines estrogenic potency and therefore also the propensity for and concentration of endocrine disruption for any lotion, creme, paste, spray, etc. for cosmetic use with or without suncare protection.

The use of silicone oils or other SPF boosting agents, such as the cocoate esters, are believed to be useful in providing SPF values of 30 or higher. The well known and commercially available “SPF boosters” have almost without exception proven to be toxic or endocrine disrupters or both and the present embodiments include a scientifically accepted and peer reviewed method to assure the use of only SPF boosters that are neither toxic nor endocrine disrupters.

### Suitable Testing for SPF Values

Testing for SPF is well known and involves either in-vivo or in-vitro methods. The in-vitro methodology is less commonly accepted and includes the use of instrumentation that delivers certain focused wavelengths of UV light to artificial skin or other substrates and measures absorbance or reflectance. In-vivo measurements are still most common and includes normally a patch test where humans are exposed to UV light that mimics sunlight. The time it takes for the subject to receive a slight reddening of the skin is then multiplied by 10 to determine the SPF value.

### Suitable Testing for Cytoprotectiveness

Testing for cytoprotectiveness can be accomplished by numerous methods including cytokine testing. One such example involved correlation with a reduction in the level of measurable nitrotyrosine. A powerful oxidant peroxynitrite (PN) from the reaction of superoxide anion with nitric oxide found catalysts to be cytoprotective against endogenously generated PN in endotoxin-stimulated RAW 264.7 cells as well as in dissociated cultures of hippocampal neurons and glia that had been exposed to cytokines. Studies thus provide compelling evidence for the involvement of peroxynitrite in cytokine-mediated cellular injury and suggest the therapeutic potential of PN decomposition catalysts in reducing cellular damage at sites of inflammation. Testing for cytoprotectiveness of suncare products such as the compositions and formulations included in this disclosure could be similarly performed to determine if skin cell damage is reduced or eliminated.

1. A formulation comprising combining; (a) at least one inorganic sun-blocking or sunscreen agent that is not endocrine disrupting, (b) at least one emollient that is not endocrine disrupting; and (c) an oil component capable of protecting skin from harmful effects of radiation including sunlight and ultraviolet light wherein said oil is a carrier oil, an essential oil, or both.

2. The formulation of claim 1, wherein said emollient consists essentially of aloe, water, and vegetable derived glycerine.

3. The formulation of claim 1, wherein the emollient comprises aloe barbadensis Miller or a single species of aloe barbadensis Miller, or aloe barbadensis Miller-Stockton present in a concentration of at least 5% by either weight or volume to enhance skin immunocompetency when applied to the skin and further comprising one or more additional components selected from sunless tanning agents, anti-microbial agents, de-pigmentation agents, anti-aging agents, anti-fungal agents, insect repellents and combinations thereof and wherein the inorganic sun-block agents are selected from the group consisting of titanium dioxide, zinc oxide, silica, silicon dioxide, fluoropolymers, and mixtures thereof, and said inorganic agents are micronized.

4. (canceled)

5. (canceled)

6. The formulation of claim 1, wherein the inorganic sun-block agents have a primary particle size of less than about 30 nm.

7. The formulation of claim 6, wherein the emollient is a salt of a not endocrine disrupting fatty acid.

8. The formulation of claim 1, wherein the formulation has a pH has a range of about 5 to about 8.5.

9. The formulation of claim 1 having a Sun Protection Factor (SPF) of at least 10, an immuno-responsiveness factor (IRF) of greater than zero, and a non-endocrine disrupter (NED) factor not greater than one, wherein the range of 0-1 is defined as a β-estradiol equivalent of less than 10 ng/g as defined by the Lumi-Cell™ test method.

10. A composition comprising a combination of: (a) at least one organic non-endocrine disrupting sunscreen agent; (b) an optional not endocrine disrupting emollient or mixtures thereof; and; (c) an optional inorganic, not-endocrine disrupting sun-block agent and; (d) an optional essential oil component comprising a carrier oil or an essential oil or both derived from an earth grown substance comprising no known endocrine disruptive agents; said composition capable of protecting skin from harmful effects of radiation including ultraviolet light and/or sunlight.

11. The composition according to claim 10, wherein said sunscreen agent is butyl-methoxydibenzoylmethane, said emollient is aloe barbadensis Miller or a single species of aloe or aloe barbadensis Miller-Stockton that includes high concentrations of oligosaccharides of aloe barbadensis Miller that enhance skin immunocompetency.

12. The composition according to claim 10, wherein active sunscreen agents, emollients, and carrier oils may include other not endocrine disruptive agents comprising a sunless tanning agent, an anti-microbial agent, a de-pigmentation agent, an anti-aging agent, an anti-fungal agent, and an insect repellent or any combination thereof, and; wherein one or more of the agents are topically active.

13. The composition according to claim 10, wherein said inorganic sun-block agents are selected from the group consisting of titanium dioxide, zinc oxide, silica or silicon dioxide or mixtures thereof and wherein said inorganic agents are micronized and wherein said inorganic agents have a primary particle size of less than about 30 nanometers.

14. (canceled)

15. The composition according to claim 10, wherein said emollient is a salt of a fatty acid, where said salt of said fatty acid has been determined to be not endocrine disrupting.

16. The composition according to claim 10, wherein said composition has a pH range of 5 to about 8.5.

17. The composition according to claim 10, having a Sun Protection Factor (SPF) of at least 10, an immuno- responsiveness factor (IRF) of greater than zero, and a non-endocrine disrupter (NED) factor not greater than one, wherein the range of 0-1 is defined as a β-estradiol equivalent of less than 10 ng/g as defined by the Lumi-Cell™ test method.

18. A sunblock or sunscreen composition comprising cold pressed aloe vera gel, deionized water, micronized zinc oxide, vegetable derived glycerin, beeswax, ascorbic vitamin C palmitate, an enzyme concentrate, rose oil, tocopheryl vitamin E acetate, retinyl vitamin A palmitate, ergocalciferol (vitamin D), xanthan gum, magnesium silicate, grapefruit seed extract, rosemary extract, cinnamon extract, and bearberry extract.

19. A sunblock or sunscreen composition comprising; (a) a cyto-protective agent derived from an earth grown substance as determined by cytokine testing; and a composition containing at least one or more of the following additional substances; (b) sunflower oil, vegetable derived glycerin, coconut oil, stearic acid as extracted from vegetable fat, beeswax, orange wax, tocopheryl acetate, buttermilk powder, sodium borate, xanthan gum, honey, sucrose stearate, glucose, glucose oxidase, lactoperoxidase, and rosemary extract and an essential oil containing fragrance and wherein the composition includes blending and mixing with a zinc oxide or other suitable inorganic particles pre-dispersed in aloe barbadensis Miller and vegetable derived glycerine and SPF boosters to ensure SPF values greater than or equal to 15.

20. A UV-protective composition for topical administration, comprising:
(a) a sunscreen or sunblocking agent selected from zinc oxide, titanium dioxide, butyl-methoxydibenzoylmethane, and mixtures thereof, in an effective amount to provide a desired level of UV protection:
(b) glucose-rich mannose-containing oligosaccharides obtained from cold processed aloe barbadensis Miller, wherein the oligosaccharides are present in combination with glycerol,
(c) a component selected from the group consisting of sunflower oil, vegetable glycerin, coconut oil, stearic acid as extracted from vegetable fat, beeswax, orange wax, tocopheryl acetate, buttermilk powder, sodium borate, xanthan gum, sucrose stearate, glucose, glucose oxidase, lactoperoxidase, rosemary extract, and essential oil-containing fragrances;
(d) enzymes or enzyme mixtures that stabilize the composition by retarding or eliminating bacteria growth, and
(e) sufficient water to form an emulsion wherein, if zinc oxide or titanium oxide is present, it is in the form of a blended dispersion with aloe barbadensis Miller, and, optionally, vegetable derived glycerine and; wherein said composition further comprises SPF boosters to ensure SPF values greater than or equal to 15.

21. The composition of claim 20 wherein said composition is formulated into a solid, a liquid, an aerosol, a cream, a lotion, an ointment, a microemulsion, a solution, or a gel form.

22. The composition of claim 20, wherein said suncreen agent comprises not endocrine disrupting agents consisting of butyl-methoxydibenzoylmethane and/or other dibenzoyl ethers and wherein any or all the agents are immunoenhancing and/or cytoprotective.

23. (canceled)

24. A UV-protective composition for topical administration, comprising;
at least one sun-block or sunscreen active agent in an amount effective to protect the skin against actinic radiation from sunlight;
(a) agents of the UV-protective compositions free of any known or suspected endocrine disrupters;
(b) a non-endocrine disruptive, cytoprotective mixture made of earth grown substances, the mixture comprising glucose-rich mannose-containing oligosaccharide or oligosaccharides obtained from and used with aloe barbadensis Miller processed at or below room temperature within 45-90 minutes of harvesting, and; optionally components including;
(c) one or more components selected from the group consisting of amino acids, vitamins or provitamins, nucleoderivatives, and vegetable extracts, wherein the amino acids comprise kyptophan, histidine, phenylalanine, tyrosine, the vitamins and provitamins comprising vitamin B6vitamin A, vitamin E, tocopherols, betacarotene, bioflavonoids, nucleotides and polymers thereof, cascara, frangula, camomile, hyperic, calendula, elicriso, licorice or essential oils thereof, and;
(d) water to form a well mixed emulsion and;
(e) wherein the composition includes blending and mixing with a zinc oxide or other suitable inorganic particles dispersed in aloe barbadensis Miller and glycerine and optionally SPF boosters to ensure SPF values greater than 15.

25. A UV-protective composition for topical administration, comprising;
(a) at least one sunscreen or sun-block active agent to provide a desired amount of UV protection;
(b) agents of the UV protective compositions free of any known or suspected endocrine disrupters;
(c) a not endocrine disrupting, cytoprotective mixture, the mixture comprising a glucose-rich mannose-containing oligosaccharide or oligosaccharides obtained from and used with aloe barbadensis Miller that can function as the at least one emollient, and;
(d) water to form a well mixed emulsion and
(e) wherein the composition includes blending and mixing with a zinc oxide or other suitable inorganic particles dispersed in aloe barbadensis Miller and glycerine and optionally SPF boosters to ensure SPF values greater than or equal to 15.

26. A UV-protective composition for topical administration, comprising;
(a) adding optionally de-ionized water, undiluted cold pressed aloe barbadensis of a single species, and zinc oxide or titanium dioxide or silicon dioxide or silica or each individually or all in any combination to a vessel;
(b) then, adding a carrier oil;
(c) then, mixing the resultant composition in said vessel.

27. The method of claim 26, comprising the steps of;
(a) adding de-ionized water, cold pressed aloe, and zinc oxide or titanium dioxide or silicon dioxide or silica or each individually or all in any combination to a vessel;
(b) then, adding a carrier oil and an emollient to said vessel;
(c) then, mixing the resultant composition;
(d) maintaining (a)-(c) at or below 80 F then, maintaining or adjusting the pH of the composition to above 5 and further comprising the steps of;
(e) adding cold pressed aloe, and zinc oxide or titanium dioxide or both in combination to a vessel;
(f) then, adding a carrier oil and an emollient and a thickener to said vessel;
(g) then, mixing the resultant composition;
maintaining (a)-(c) at or below 80 F then, maintaining or adjusting the pH of the composition to above 5.

28. (canceled)

29. A composition comprising sunscreen compositions containing sunscreen agents that provide non-endocrine disruptive, adequate, safe protection for mammalian skin while also enhancing the skin's immuno-responsiveness from cancerous or pre-cancerous skin cells in the presence of radiation such as UV light or sunlight.

30. A dispersion composition comprising an “all-natural” and primarily all earth grown ingredient based dispersion of inorganic sunblocking agents that will ensure an SPF value of at least 15 or greater and wherein the dispersion must not have any endocrine disrupting agents or known toxins within said dispersion and wherein said dispersion is used to complete a sunblocking or sunscreen composition and wherein a test method for determining whether there are any endocrine disrupting ingredients, active or inactive, in a sunscreen or sun-block composition or any other composition used for sun protection, skin care, hair care, wherein said test method is the LUMI-CELL™ method.

31. (canceled)

32. A test method using Applied-Kinesiology, comprising determining a response that any skin care composition has on a user's neuro-muscular strength, wherein the neuro-muscular testing diagnosis using Applied-Kinesiology is determining a composition's effect upon neuro-muscular response of a human exposed to said composition or any combination of compositions together with a LUMI-CELL™ test method, to ensure that all ingredients of said composition, including inactive and active ingredients used in said skin care composition are non-endocrine disrupting, non-toxic, and/or immunoenhancing, wherein a non-endocrine disrupter (NED) factor is determined to be not greater than one, wherein the range of 0-1 is defined as a β-estradiol equivalent of less than 10 ng/g as defined by said Lumi-Cell™ test method.
